(19) United States
(12) Patent Application Publication
(10) Pub. No.: US 2008/0262615 A1
(43) Pub. Date: Oct. 23, 2008

(54) Digital Joint Arthroplasty

(75) Inventors: Malan de Villiers, Irene (ZA); Ulrich Mennen, Waterkloof (ZA)

Correspondence Address: PAULEY PETERSEN & ERICKSON, 2800 WEST HIGGINS ROAD, SUITE 365, HOFFMAN ESTATES, IL, 60195, US

(73) Assignee: Graham Alan Blackbeard, Irene (ZA)

(21) Appl. No.: 11/629,455

(22) PCT Filed: Jun. 23, 2005
(86) PCT No.: PCT/IB05/01780

§ 371 Date: Feb. 13, 2008

(30) Foreign Application Priority Data
Jun. 23, 2004 (ZA) 2004/4961

(51) Int. Cl. A61F 2/08; A61F 2/42

(52) U.S. Cl. 623/14.12; 623/21.11

(57) ABSTRACT

The invention concerns a digital joint arthroplasty (10). The arthroplasty has a stemless, unitary body having opposite sides (12, 14) at least one of which is formed with a curved concavity shaped to receive, in articulated manner, a bone end in an MCP, PIP or DIP joint. In some embodiments, both of the opposite side surfaces are concavely curved while in other embodiments, only one side surface (12) is so curved, the opposite surface (40) being flat. In use the arthroplasty is installed between the shaped ends of bones which meet at the MCP, PIP or DIP joint. The absence of stems means that the installation of the arthroplasty is less surgically invasive than is the case with conventional arthroplasties having inter-medullary stems.

### BACKGROUND TO THE INVENTION

THIS invention relates to a digital joint arthroplasty.

Arthritis of the digital or interphalangeal joints is a painful condition arising from bone-on-bone contact during articulation of the metacarpal bone against the proximal phalangeal bone or proximal phalanx in the case of the metacarpophalangeal or MCP joint, the proximal phalangeal bone against the middle phalangeal bone or phalanx in the case of the proximal interphalangeal or PIP joint, or the middle phalangeal bone against the distal phalangeal bone or distal phalanx in the case of the distal interphalangeal or DIP joint. Progression of the disease can also lead to misalignment of the bones at these joints.

Known treatments for arthritis include debridement of the articulating surfaces, excision or fusion of the affected joint or replacement of the joint with a prosthesis or arthroplasty.

One currently used arthroplasty, known as the Swanson prosthesis, has a central, generally U-shaped hinge element and a pair of oppositely projecting intramedullary stems which are in use seated in holes drilled for the purpose in the ends of the bones which meet at the joint. The device is made of a flexible silicone. Apart from the danger of breakage or fracture, it has been observed that the silicone may be abraded with the result that silicone particles are produced. Such particles may cause irritation to, or even destruction of, the surrounding tissue.

Another currently used arthroplasty has two components with cooperating convex and concave surfaces which articulate against one another in the joint. The components are attached to their respective bones by rigid intramedullary stems which seat in holes formed in the ends of the bones. In some cases, a single component is fixed to one bone end to articulate against a shaped end of the other bone. In such arrangements it is possible for the stem(s) to work loose, compromising the effectiveness of the arthroplasty.

Primarily because of their use of intramedullary stems, both types of known arthroplasty involve invasive surgical procedures and may cause damage to adjacent ligamentous structures. In addition, where there is a tendency for the stem(s) to work loose, the arthroplasty has a limited life expectancy. Frequently, failure of the arthroplasty leaves no option other than fusion of the joint.

The present invention seeks to provide a digital joint arthoplasty the insertion of which is less invasive than is the case with conventional arthroplasties and which, it is believed, will provide comparable or increased longevity.

### SUMMARY OF THE INVENTION

According to the present invention there is provided a digital joint arthroplasty comprising a stemless, unitary body having opposite sides at least one of which is formed with a curved concavity shaped to receive, in articulated manner, a bone end in an MCP, PIP or DIP joint.

Preferably, a concavity in one side of the body has a spherical curvature. In some embodiments, the opposite side of the body may be formed with a concavity having a spherical or cylindrical curvature. In other embodiments, only one side of the body is formed with a concavity, the opposite side being at least partially flat.

In embodiments where both sides of the body are formed with concavities, the concavity on one side of the body may be deeper and have a greater radius of curvature than the concavity on the opposite side of the body.

Other features of the invention are set forth in the appended claims.

### BRIEF DESCRIPTION OF THE DRAWINGS

The invention will now be described in more detail, by way of example only, with reference to the accompanying drawings in which:

FIG. 1 shows a perspective view of a digital joint arthroplasty according to a first embodiment of this invention;

FIG. 2 shows another perspective view of the arthroplasty of FIG. 1;

FIG. 3 shows an elevation of the arthroplasty of FIG. 1;

FIG. 4 shows a side view of the arthroplasty of FIG. 1;

FIG. 5 shows a plan view of the arthroplasty of FIG. 1;

FIG. 6 shows a perspective view of a digital joint arthroplasty according to a second embodiment of the invention;

FIG. 7 shows another perspective view of the arthroplasty of FIG. 6;

FIG. 8 shows an elevation of the arthroplasty of FIG. 6;

FIG. 9 shows a side view of the arthroplasty of FIG. 6; and

FIG. 10 shows a plan view of the arthroplasty seen in FIG. 6.

### DESCRIPTION OF THE ILLUSTRATED EMBODIMENTS

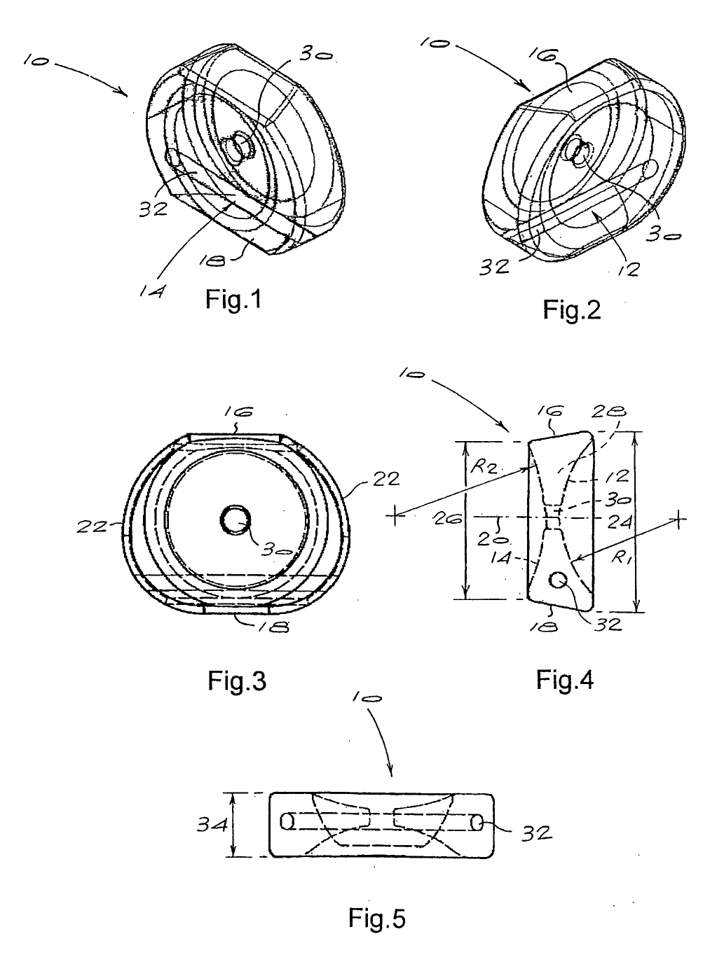

The digital joint arthroplasty illustrated in FIGS. 1 to 5 is designed for use in an MCP, PIP or DIP finger joint. In this application, the arthroplasty is placed between the ends of the relevant bones of the joint.

The arthroplasty consists of a unitary body 10 made in this embodiment of cross-linked, medical grade UHMWPE (ultra high molecular weight polyethylene). In other embodiments of the invention, the body could be made of other suitable materials, including other medical grade polymers or metals. The body 10 has opposite sides formed with opposing, spherical concavities 12 and 14 with respective radii of curvature R₁ and R₂. In practice, the ends of the bones which meet at the joint in question are appropriately shaped, during the surgical procedure, to fit into the concavities in an articulating manner.

The overall shape and dimensions of the body 10 are selected for insertion into the specific joint in question. Taking a PIP joint as an example, the concavity 12 is designed to receive the end of the larger of the two bones, i.e. the proximal bone, while the concavity 14 is designed to receive the end of the smaller of the two bones, i.e. the distal bone. For this reason, the concavity 12 is somewhat deeper than the concavity 14 and the radius R₁ is typically slightly greater than the radius R₂.

The body 10 has operatively upper and lower external edge surfaces 16 and 18 which are planar, convergent and inclined relative to the axis 20 of the body. The other external edge surfaces 22 of the body, which extend between the upper and lower edge surfaces 16 and 18, are curved as shown. It will be noted that the surface 16 is narrower than the surface 18.

It will be understood that the dimensions of the edge surfaces 16 and 18 are selected to suit the dimensions of the upper and lower surfaces of the proximal and distal bones which meet at the joint. Referring to FIG. 4 in particular it will also be understood that the dimensional relationship of the surfaces 16 and 18, together with their inclinations, results in a structure in which the dimension 24 is somewhat greater than the dimension 26. This provides extra space for the deeper concavity 12 and for that concavity to have a slightly larger radius of curvature than the concavity 14.

Between the bases of the concavities 12 and 14 there is a web 28 of material which is pierced centrally by an opening 30 on the axis of the body. In addition to the hole 30, the body 10 is pierced by a transverse suture passage 32. The functions of the opening 30 and passage 32 are described below.

In use, the ends of the bones which meet at the joint in question are, as indicated above, shaped to fit into the concavities 12 and 14. The body 10 is then inserted between the bone ends with those ends received in the concavities. The body is retained in place by the tension across the joint applied by the surrounding ligamentous structures, and it will be understood that the overall thickness 34 of the body 10 and of the web 28 are selected in each application to ensure optimal tension in these structures.

Should there be a requirement for extra security against lateral displacement of the body 10 a retaining suture can be passed through the passage 32 and around or otherwise into engagement with the adjacent volar ligament. This may not always be required, so the passage 32 is an optional feature which is not necessarily included in all embodiments of the invention.

In surgery involving a digital joint it is often the practice to immobilise the affected joint by means of a temporary, so-called K-wire (Kirschner wire) which is passed longitudinally through the bones and the joint. After healing of the tendons and/or ligaments the K-wire is withdrawn.

With the present embodiment, the K-wire can be passed longitudinally through the bones and through the opening 30 in the web 28. Once again, the K-wire may not be used in all applications, so the opening 30 is an optional feature.

An important feature of the illustrated embodiment is the fact that the body 10 has no intermedullary stems to locate in holes formed in the ends of the bones. To the extent that there is accordingly no requirement to drill the bones to receive the stems, a surgical procedure involving the insertion of an arthroplasty according to the present invention may be considered to be less surgically invasive. The absence of medullary stems also means that the possibility of such stems loosening with passage of time with possible, resultant reduction in longevity is eliminated. It is also noted that the use of the arthroplasty of the present invention will typically only involve moderate or even minimal reshaping of the ends of the bones so, once again, the surgical procedure may be seen to be relatively non-invasive.

In practice, the end of the proximal bone in a digital joint has a generally convex shape while the adjacent end of the distal bone is slightly concave to receive, in a healthy joint, the end of the proximal bone. The concavity at the end of the distal bone may require fairly substantial reshaping during the surgical procedure to provide it with a convex shape suitable to seat in the concavity 14. In some situations, reshaping of the bone to the extent required may be undesirable.

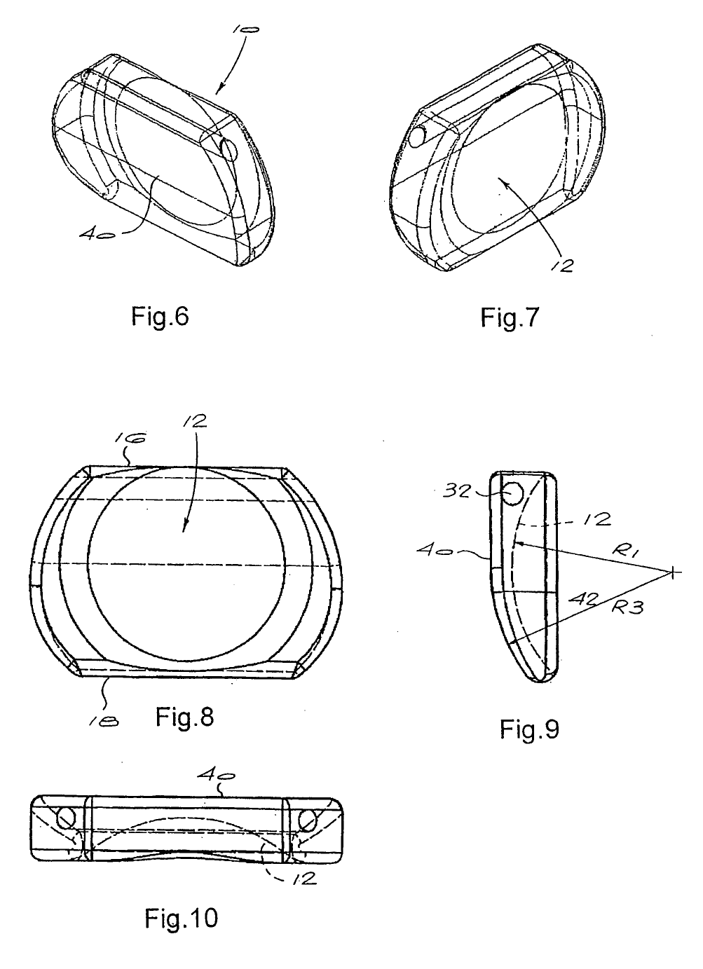

FIGS. 6 to 10 illustrate a second embodiment of invention which addresses this potential problem. In these Figures components corresponding to those seen in FIGS. 1 to 5 are designated by the same reference numerals.

The main difference between the second embodiment and the first embodiment is the fact that the body 10 is formed with a single concavity 12, of spherical shape and radius of curvature R₁, to receive the end of the proximal bone in the digital joint.

The opposite surface of the body 10, designated 40, is flat. In other embodiments (not illustrated), this surface may even be convex.

In use, the arthroplasty of the second embodiment is placed between the adjacent ends of the proximal and distal bones as described above for the first embodiment. The end of the proximal bone may be moderately shaped, if necessary, for reception by the concavity 12. The end of the distal bone may if necessary be shaped to seat on the opposite surface 40. Although there is no positive reception of the end of the distal bone in a concavity as in the first embodiment, the ligamentous structures surrounding the joint will retain the arthroplasty 10 in position. In addition, as in the first embodiment, a suture may be passed through the suture passage 32 and engaged with the volar ligament to provide additional security against displacement of the arthroplasty.

An advantage of the second embodiment is the fact that minimal shaping of the end of the distal bone is required for it to seat against the flat (or convex) surface 40.

As will be apparent from FIGS. 6 to 10, the arthroplasty tapers downwardly with an externally spherical, convex curvature R₃.

As with the first embodiment, the dimensions of the arthroplasty are selected to suit the particular digital joint under consideration. The thickness of the arthroplasty may, for instance, be 3 mm, 4 mm or 5 mm.

In the second embodiment, the surface 40 is flat. It is however within the scope of the invention for this surface to be convex.

In the above description and appended claims, terms such as “upper”, “lower” and so forth are with reference to a finger joint with the finger itself extended laterally, knuckle upwards.

1-16. (canceled)

17. A digital joint arthroplasty comprising:
a stemless unitary body having opposite sides, at least one side of the opposite sides forming a curved concavity shaped to articulatingly receive a bone end in one of a metacarpophalangeal joint, a proximal interphalangeal joint and a distal interphalangeal joint.

18. The digital joint arthroplasty of claim 17 wherein the curved concavity has a spherical curvature.

19. The digital joint arthroplasty of claim 17 wherein the curved concavity has a cylindrical curvature.

20. The digital joint arthroplasty of claim 17 wherein the stemless unitary body is made of one of a medical grade polymer and a medical grade metal.

21. The digital joint arthroplasty of claim 20 wherein the medical grade polymer is an ultra high molecular weight polyethylene.

22. The digital joint arthroplasty of claim 17 wherein each of the opposite sides of the stemless unitary body forms a curved concavity.

23. The digital joint arthroplasty of claim 22 wherein the curved concavity in one side of the stemless unitary body has a spherical curvature and the curved concavity in the opposite side of the stemless unitary body has one of a spherical curvature and a cylindrical curvature.

24. The digital joint arthroplasty of claim 22 wherein the curved concavity in one side of the stemless unitary body is deeper than the curved concavity in the opposite side of the stemless unitary body.

25. The digital joint arthroplasty of claim 24 wherein the deeper curved concavity has a radius of curvature greater than a radius of curvature of the curved concavity in the opposite side of the stemless unitary body.

26. The digital joint arthroplasty of claim 22 wherein the stemless unitary body further comprises an opening extending through the stemless unitary body and through bases of the curved concavities.

27. The digital joint arthroplasty of claim 17 wherein one side of the opposite sides of the stemless unitary body forms the curved concavity and an opposite side is partially flat.

28. The digital joint arthroplasty of claim 17 wherein one side of the opposite sides of the stemless unitary body forms the curved concavity and an opposite side is at least partially convex.

29. The digital joint arthroplasty of claim 17 wherein one side of the opposite sides of the stemless unitary body forms the curved concavity and an opposite side is partially flat and partially convex.

30. The digital joint arthroplasty of claim 17 wherein the stemless unitary body further forms a generally planar upper edge surface and a generally planar lower edge surface opposite the upper edge surface.

31. The digital joint arthroplasty of claim 30 wherein the upper edge surface and the lower edge surface lie in convergent planes, the convergent planes inclined relative to an axis of the stemless unitary body.

32. The digital joint arthroplasty of claim 30 wherein the stemless unitary body further forms curved side edges extending between the upper edge surface and the lower edge surface.

33. The digital joint arthroplasty of claim 17 wherein the stemless unitary body forms a suture passage extending therethrough.

34. The digital joint arthroplasty of claim 33 wherein the stemless unitary body forms an upper edge surface and a lower edge surface opposite the upper edge surface, the suture passage extending through the stemless unitary body parallel to the upper edge surface and the lower edge surface.

35. A digital joint arthroplasty comprising:
a stemless unitary body having opposite sides, at least one side of the opposite sides forming a curved concavity shaped to articulate a bone end in one of a metacarpophalangeal joint, a proximal interphalangeal joint and a distal interphalangeal joint; and
a suture passage extending through the stemless unitary body.

36. A digital joint arthroplasty comprising:
a stemless unitary body having opposite sides, each of the opposite sides forming a curved concavity having a base and shaped to receive a bone end of one of a metacarpophalangeal joint, a proximal interphalangeal joint and a distal interphalangeal joint;
the stemless unitary body defining a generally planar upper edge surface and a generally planar lower edge surface opposite the upper edge surface, the upper edge surface and the lower edge surface lying in convergent planes extending between and connecting the opposite sides and inclined relative to an axis of the stemless unitary body;
an opening formed through the stemless unitary body at bases of curved concavities; and
a suture passage extending through the stemless unitary body generally parallel to the upper edge surface and the lower edge surface.
